SEQUENCE LISTING (6 sequences)

SEQ ID NO: 1 - DNA, 151 bp, Homo sapiens
  source 1..151: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcac

SEQ ID NO: 2 - protein, 590 aa, Homo sapiens
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 3 - protein, 590 aa, Homo sp.; identical to SEQ ID NO: 2

SEQ ID NO: 4 - protein, 289 aa, Mus musculus
MTGVFDRRVPSIRSGDFQAPFPTSAAMHHPSQESPTLPESSATDSDYYSPAGAAPHGYCSPTSASYGKALNPYQYQYHGVNGSAAGYPAKAYADYGYASPYHQYGGAYNRVPSATSQPEKEVAEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYPSAASSINSHLPPPGSLQHPLALASGTLY

SEQ ID NO: 5 - protein, 184 aa, Mus musculus
MTGVFDRRVPSIRSGDFQAPFPTSAAMHHPSQESPTLPESSATDSDYYSPAGAAPHGYCSPTSASYGKALNPYQYQYHGVNGSAAGYPAKAYADYGYASPYHQYGGAYNRVPSATSQPEKEVAEPEVRMVNGKPKKVRKPRTIYSSYLENSASWYPSAASSINSHLPPPGSLQHPLALASGTLY

SEQ ID NO: 6 - protein, 146 aa, Mus musculus
MTGVFDRRVPSIRSGDFQAPFPTSAAMHHPSQESPTLPESSATDSDYYSPAGAAPHGYCSPTSASYGKALNPYQYQYHGVNGSAAGYPAKAYADYGYASPYHQYGGAYNRVPSATSQPAFSWPLYREGFRRLSTSPCQNARSWPPL

(19) United States
(12) Patent Application Publication
(10) Pub. No.: US 2024/0100190 A1
(43) Pub. Date: Mar. 28, 2024

(54) Brain Disease Therapy Enhancing Inhibition of GABAergic Neurons

(71) Applicant: City University of Hong Kong, Kowloon (HK)

(72) Inventors: Jufang He, Kowloon (HK); Ling He, Kowloon (HK); Yujie Yang, Kowloon (HK); Junming Ren, Kowloon (HK)

(21) Appl. No.: 17/935,686

(22) Filed: Sep. 27, 2022

(51) Int. Cl. A61K 48/00; A61K 38/17; A61P 25/08; C12N 15/86

(52) U.S. Cl. CPC A61K 48/0066; A61K 38/177; A61P 25/08; C12N 15/86; C12N 2750/14143; C12N 2750/14145

(57) ABSTRACT

A method for a method of prophylaxis and/or therapy of neurological conditions in a subject in need thereof includes the step of administrating a recombinant AAV vector to the subject. Also disclosed is the use of the a recombinant AAV vector in the manufacture of a medicament of prophylaxis and/or therapy of neurological conditions in a subject in need thereof. The recombinant AAV vector includes: PHP.eB; a sequence for introducing the expression of human M3 muscarinic DREADD receptor coupled to Gq (hM3Dq); and an inhibitory neuron-specific Distal-less homeobox (Dlx) gene enhancer sequence.

### SEQUENCE LISTING

The Sequence Listing file entitled “sequencelisting” having a size of 7,804 bytes and a creation date of Sep. 27, 2022, that was filed with the patent application is incorporated herein by reference in its entirety.

### FIELD OF THE INVENTION

The present invention generally relates to a new therapy for brain diseases or disorders, particularly a therapy of epilepsy, by balancing the inhibition and excitation in the brain.

### BACKGROUND

Epilepsy is one of the most common neurological diseases and affects people of all ages, races, social classes, and geographical locations. According to the World Health Organization, around 50 million people had epilepsy in 2019, with an increment of 2 million yearly. However, access to effective anti-seizure treatment remains out of reach for most people with epilepsy, particularly in lower and middle-income countries. The estimates of the treatment gap in low-income countries are over 75% and tend to be higher in rural versus urban areas (R. A. Scott, et al., Policy and Practice The treatment of epilepsy in developing countries: where do we go from here? World Health, Vol. 79, pp. 344-351, 2001). Thus, providing therapy with long-term medical efficiency for patients suffering from epilepsy is urgently needed.

The causes of epilepsy can be classified into genetic (i.e., genetic channelopathies/malformations), cryptogenic (the cause is hidden), and other (i.e., head trauma, brain tumors and strokes, autism, or neurofibromatosis (NF), and meningitis, viral encephalitis). It is believed that those causes lead to an imbalance in the excitatory and inhibitory activities of the brain. Accordingly, most current treatment options relate to restoring the brain's lost excitation/inhibition balance. The main therapeutic methods focus on the levels of excitatory/inhibitory neurotransmitters or neuropeptides within the synaptic cleft. Current medications to suppress seizure onset apply exogenous reagents to enhance inhibitory synaptic transmission and block spike generation. After treatment, many epileptic patients become insensitive to original medications while the elevated ratio of cortical excitation to inhibition pathogenesis is still present. The reasons for lack of long-term medical efficiency may include that the medical treatment is not neuron-specific, and the seizure self-termination has not been considered. Therefore, there remains a need to develop new therapeutic strategies against epilepsy based on strengthening the endogenous mechanisms of seizure self-termination in a neuron-specific manner.

Gene therapies have attempted to address neurological disorders that distribute widely in the brain or involve the entire nervous system, such as Alzheimer's disease, Parkinson's disease (Hudry, et al., Neuron, Vol. 101, 839-862, 2019), Huntington's disease (Hocquemiller, et al., Hum. Gene Ther., Vol. 27, pp. 478-496, 2016), and Duchenne muscular dystrophy (Zhang, et al., Sci. Adv., Vol. 6, pp. 1-12, 2020). The Adeno-associated Virus' (AAVs') efficient and targetable properties provide a long-term and safe expression in the nervous system (George, et al., Mol. Ther., Vol. 28, pp. 2073-2082, 2020; Nguyen, et al., Nat. Biotechnol., Vol. 39, 47-55, 2021 Samulski, et al., Annu Rev Virol, Vol. 1, pp. 427-51, 2014). Despite recent advances in the development of AAV-based gene therapy for neural disorders (George, et al., 2020; Hocquemiller, et al., 2016; Hudry, et al., 2019; Nguyen, et al., 2021; Samulski, et al., 2014; Zhang, et al., 2020), both the clinical trials and preclinical studies face several obstacles (Hudry, et al., 2019), including the therapeutic potency, AAV vector immunogenicity (Ronzitti, et al., Front. Immunol., Vol. 11, pp. 1-13, 2020; Verdera, et al., Mol. Ther., Vol. 28, pp. 723-746, 2020), and potential genotoxicity (Chandler, et al., J. Clin. Invest., Vol. 125, pp. 870-880, 2015). Thus, there remains a need to develop new AAV vectors for targeting specific neurons, which are safer and more efficient but with minimized side effects. For example, U.S. Ser. No. 10/682,424 B2 relates to a recombinant AAV vector comprising an I56i enhancer sequence, and a sequence encoding hM3Dq modified muscarinic receptor (Gq-DREADD). However, it appears that the virus should be delivered directly to the target brain areas with at least one injection or multiple injections after craniological surgery, which may not be convenient and safe for epilepsy patients.

Refractory epilepsy is difficult to cure due to the lack of specific therapeutic targets (Kanter-Schlifke, et al., Mol. Ther., Vol. 15, pp. 1106-1113, 2007; Wang, et al., Mol. Ther., Vol. 27, pp. 2018-2037, 2018). Epileptogenesis shares a key property of a combination of hyperactivity and hyper synchronization (Colasante, et al., Brain, Vol. 143, pp. 891-905, 2020). Dysfunctions of GABAergic neurons or their receptors were found in epilepsy (Gallo, et al., Trends Neurosci., Vol. 43, pp. 565-580, 2020; Shore et al., Cell Rep., Vol. 33, p. 108303, 2020). The relatively small percentage of inhibitory GABAergic neurons that account for <15˜20% of the neocortical neurons play a vital role in maintaining the excitation and inhibition balance in the brain (Berg, et al., Science (80-.), Vol. 315, pp. 390-393, 2007; Sun, et al., Nature, Vol. 465, pp. 927-931, 2010; Xue, et al., Nature, Vol. 511, pp. 596-600, 2014). The following strategies have been proposed for enhancing the inhibitory influences of GABAergic neurons: 1) the potentiation of inhibitory outputs through releasing more GABA transmitters (Fiszman, J. Neurosci., Vol. 25, pp. 2024-2031, 2005), 2) increasing PV expression (Favuzzi, et al., Neuron, Vol. 95, pp. 639-655, 2017), and 3) selective gating of glutamatergic inputs by presynaptic GABAergic inhibition (Pan, et al., J. Pharmacol. Exp. Ther., Vol. 331, pp. 591-597, 2009). However, the need remains for a method or therapy for obtaining long-lasting potentiated inhibition.

### SUMMARY OF THE INVENTION

An embodiment of the present invention relates to a method of prophylaxis and/or therapy of neurological conditions in a subject in need thereof comprising the step of administrating a recombinant AAV vector to the subject, in which the recombinant AAV vector targets GABAergic neurons and includes: PHP.eB; a sequence for introducing the expression of human M3 muscarinic DREADD receptor coupled to Gq (hM3Dq); and an inhibitory neuron-specific Distal-less homeobox (Dlx) gene enhancer sequence.

An embodiment of the present invention relates to a use of a recombinant AAV vector in the manufacture of a medicament of prophylaxis and/or therapy of neurological conditions in a subject in need thereof, in which the recombinant AAV vector includes: PHP.eB; a sequence for introducing the expression of human M3 muscarinic DREADD receptor coupled to Gq (hM3Dq); and an inhibitory neuron-specific Distal-less homeobox (Dlx) gene enhancer sequence.

Without intending to be limited by theory it is believed that the present invention may provide one or more advantages such as enhanced inhibition of GABAergic neurons, long term safe and stable therapeutic effects, and minimized side effects during and after the introduction of the vector.

### BRIEF DESCRIPTION OF THE DRAWINGS

FIG. 1A is a series of plots showing GABAergic neuron inhibition before and after Low-Frequency Laser Stimulation (LFLS, 1 Hz) in the AAV9-EF1α-DIO-ChETA-eYFP injected mice group according to Example 2.

FIG. 1B and FIG. 1C are a series of graphs showing GABAergic neuron inhibition before and after High Frequency Laser Stimulation (HFLS; 40 Hz or 80 Hz) in the AAV9-EF1α-DIO-ChETA-eYFP injected mice group according to Example 2.

FIG. 2A is a diagram showing the rAAV-mDlx-hM3Dq-eGFP-PHP.eB (hereinafter referred to as hM3Dq) or rAAV-mDlx-eGFP-PHP.eB (hereinafter referred to as eGFP) injection of C57BL6J mice according to Example 3.

FIG. 2B shows confocal images of eGFP reporter gene co-staining with GAD67 in the hM3Dq-injected mice (left panels) and eGFP-injected mice (right panels; scale bars: 500 μm and 20 μm for the zoomed figures) according to Example 3.

FIG. 2C and FIG. 2D show the Local Field Potential (LFP) responses to the AS according to Example 4, in which FIG. 2C shows the representative trace before (baseline) and after (0-15 min and 105-120 min) CNO injection recorded in the eGFP-injected mice (upper panels) and hM3Dq-injected mice (lower panels); and FIG. 2D shows the time course of LFP responses to the AS before and after CNO injection (eGFP group in black; n=12 recording sites; N=8 mice; hM3Dq group in grey; n=10 recording sites; N=7 mice; two-way RM ANOVA, ****P<0.0001), and mean LFP amplitudes before (baseline) and about 2 h after CNO injection (105-120 min; one-way ANOVA, N.S.; ****P<0.0001).

FIG. 3A shows whole-cell current-clamp recording in the cortical slices (left panels, scale bars: 50 μm) injected with eGFP and hM3Dq; representative images (middle panels) for the step current-evoked firing response; and the spontaneous action potential traces (right panels) for the baseline vs. the after-CNO according to Example 3.

FIG. 3B is a diagram showing numbers of recording cells with burst firing after CNO application in the eGFP and the hM3Dq-injected mice (a burst defined as more than 3 spikes within one burst; the inter-spike interval is less than 100 ms between spikes) according to Example 3.

FIG. 4A is a diagram showing the hM3Dq and DIO-ChrimsonR-mCherry injection into the cortex of Vgat-Cre mice.

FIG. 4B shows confocal images of eGFP and mCherry reporter gene expression (scale bars: 50 μm and 20 μm for the zoomed figures).

FIG. 4C shows representative Inhibitory Postsynaptic Current (IPSC) traces before (baseline) and 35-40 min after the CNO injection, the HFLS, or the LFLS. As shown in FIG. 4C, robust potentiation of laser pulse induced IPSCs can be found either after CNO or HFLS application, while not after LFLS (waveforms of IPSC responses to the laser stimulation at 0.1 Hz, baseline and 35-40 min after the CNO, HFLS, and LFLS; the time course of normalized IPSCs amplitudes, baseline vs. 35-40 min, CNO: 100.22±0.66% vs. 250.62±6.46%; HFLS: 100.00±1.67% vs. 241.41±5.54%; LFLS: 100.01±0.25% vs. 99.74±1.28%, two-way RM ANOVA with Tukey's post hoc test, ****P<0.0001, ####P<0.0001, N.S.). Together, these results indicate that the enhancement of inhibition could be induced either by chemogenetic or optogenetic approach via high frequency firing of GABAergic neurons.

FIG. 5 is the map for the composition of rAAV-mDlx-hM3Dq-eGFP vector.

FIG. 6A shows confocal images of eGFP reporter gene co-staining with GAD67 in the hM3Dq-injected mice (left panels) and eGFP-injected mice (right panels; scale bars: 500 μm and 20 μm for the zoomed images) according to Example 5.

FIG. 6B and FIG. 6C show the LFP responses to the AS according to Example 5, in which FIG. 6B shows the representative trace before (baseline) and after (0-15 min and 105-120 min) CNO injection recorded in the eGFP-injected mice (upper panels) and the hM3Dq-injected mice (middle panels), and FIG. 6C shows the time course of LFP responses to the AS before and after CNO injection (eGFP group in black; n=11 recording sites; N=7 mice; hM3Dq group in red; n=14 recording sites; N=8 mice; two-way RM ANOVA, ****P<0.0001).

FIG. 7A and FIG. 7B show confocal images of GFP reporter gene and GAD67 co-expression across the left hemisphere of an epileptic mouse after rAAV-mDlx-hM3Dq-eGFP-PHP.eB (A) or rAAV-mDlx-eGFP-PHP.eB (B) delivery via intravenous injection (scale bars: 1000 μm) according to Example 5.

FIG. 8A shows confocal images of the intra-hippocampal KA-injected mice (upper panel for the KA-injected mice), KA and eGFP-injected mice (middle panel for the eGFP-injected mice), and KA hM3Dq-injected mice (lower panel for the hM3Dq-injected mice; scale bars: 1000 μm) according to Example 6.

FIG. 8B shows heat maps of the number of seizures detected daily in the KA-injected mice (upper panel, 10 mice), eGFP-injected mice (middle panel, 9 mice), and hM3Dq-injected mice (lower panel, 11 mice) according to Example 6.

FIG. 8C is dot charts of the average number of daily seizures per week (paired t-test, N.S., *P<0.05, **P<0.01) according to Example 6.

FIG. 8D is a bar chart of the fold change of seizures among 3 groups (one-way ANOVA with Tukey's post hoc test, N.S., *P<0.05, **P<0.01) according to Example 6.

FIG. 9A are images showing sections from the hM3Dq-injected mice according to Example 7: upper panel: overlayed image for GFAP, eGFP, and DAPI (scale bar: 500 μm); and lower panels: magnified images for GFAP and DAPI, indicated by the numbers in the upper panel (scale bars: 50 μm).

FIG. 9B are images showing sections from the eGFP-injected mice according to Example 7: upper panel: overlayed image for GFAP, eGFP, and DAPI (scale bar: 500 μm); and lower panels: magnified images for GFAP and DAPI, indicated by the numbers in the upper panel (scale bars: 50 μm).

FIG. 9C is a plot showing group data that compares active time with and between two groups mice according to Example 7 (hM3Dq-injected mice, N=11 mice; eGFP-injected mice, N=9 mice; one-way ANOVA with Tukey's post hoc test, *P<0.05; N.S., not significant).

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, 25 pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

### Definitions

As used herein, “adeno-associated virus (AAV) vector” is a single-stranded DNA virus and belongs to the Dependovirus genus of the Parvoviridae family.

As used herein, “recombinant AAV vector” refers to a vector generated by replacing the wild type AAV open reading frames with a target (therapeutic or marker) gene expression cassette.

As used herein, “DREADD” refers to Designer Receptor Exclusively Activated by a Designer Drug. As used herein, “human M3 muscarinic DREADD receptor coupled to Gq (hM3Dq)” refers to a modified muscarinic receptor that can be activated by clozapine-N-oxide (CNO) or analogue thereof with high potency and efficacy. The amino acid sequence of the hM3Dq is known in the art, and the instant invention includes all polynucleotide sequences encoding the hM3Dq. In some embodiments, the polynucleotides encoding the hM3Dq comprises an amino acid sequence according to any of SEQ ID NOs. 2-3 or a functionally equivalent sequence with an identity of from at least about 80%, at least about 90%, about 95%, about 96%, about 97%, about 98% or about 99% to about 100% of any one of SEQ ID NOs. 2-3.

As used herein, “inhibitory neuron-specific Distal-less homeobox (Dlx) gene enhancer sequence” refers to the Dlx gene family contain a homeobox that is related to Distal-less (Dll), the gene expressed in the head and limbs of the developing fruit fly. The present disclosure includes use of any Dlx enhancer sequence from any species, provided the enhancer will function to promote expression of the hM3Dq receptor. In some embodiments, the Dlx enhancer may include an amino sequence according to any one of SEQ ID NOs. 4-6 or a functionally equivalent sequence with an identity of from at least about 80%, at least about 90%, about 95%, about 96%, about 97%, about 98% or about 99% to about 100% of any one of SEQ ID NOs. 4-6.

As used herein, “GABAergic inhibitory neurons” herein include but are not limited to the GABAergic neurons expressing parvalbumin (PV), somatostatin (SOM), vasoactive intestinal peptide (VIP), cholecystokinin (CCK), neuropeptide Y (NPY), CBPs calbindin (CB), or calretinin (CR).

As used herein, GABAergic neurons produce gamma-Aminobutyric acid (GABA), which is the main inhibitory neurotransmitter in the mammalian central nervous system (CNS). GABA is primarily synthesized from glutamate, catalyzed by glutamate decarboxylase (GAD), and is present at 30-40% of synapses. GABA induces either Cl− influx or K+ efflux, resulting in hyperpolarized neurons and reduced action potential. Dysfunction of GABA neurotransmission can result in several disorders, including schizophrenia and epilepsy.

As used herein, “long-termed inhibition” refers to that the enhanced inhibition of inhibitory neurons provided by the invention lasts for at least 1 week.

As used herein, “pharmaceutically acceptable carrier” includes but is not limited to an excipient, a buffer, a stabilizer and/or a preservative that is used for pharmaceutical compositions and well known to those skilled in the art.

An embodiment of the present invention relates to a recombinant AAV vector including PHP.eB, a sequence for introducing the expression of human M3 muscarinic DREADD receptor coupled to Gq (hM3Dq); and an inhibitory neuron-specific Distal-less homeobox (Dlx) gene enhancer sequence.

Without intending to be limited by theory, it is believed that AAV vector is one of the most attractive gene transfer tools in developing novel genetic therapies for neurological diseases. The designed AAV vectors herein introduce the expression of the human M3 muscarinic DREADD receptor coupled to Gq (hM3Dq) under the control of the mDlx.

Surprisingly, the new recombinant AAV vector of the invention may, for example, specifically target inhibitory neurons, in particular GABAergic inhibitory neurons, and results in no astrocytic reactivity in cortex and no changes in locomotion after being administrated to a subject for treating brain conditions, as verified in the examples.

In an embodiment herein, the inhibitory neurons include GABAergic interneurons selected from the group consisting of parvalbumin expressing GABA neurons (PV-GABA neurons), somatostatin expressing GABA neurons (SOM-GABA neurons), vasoactive intestinal peptide expressing GABA neurons (VIP-GABA neurons), cholecystokinin expressing GABA neurons (CCK-GABA neurons), neuropeptide Y expressing GABA neurons (NPY-GABA neurons), CBPs calbindin expressing GABA neurons (CB-GABA neurons), calretinin expressing GABA neurons (CR-GABA neurons), and a combination thereof. For example, the GABAergic inhibitory neurons may be combinations of CCK-GABA neurons and VIP-GABA neurons, CCK-GABA neurons and SOM-GABA neurons, or CB-GABA neurons and SOM-GABA neurons.

An embodiment herein of the present invention relates to a composition or a medicament including the recombinant AAV vector herein.

In an embodiment herein, the composition or the medicament further includes a pharmaceutically acceptable carrier, as defined herein. In some embodiments, the compositions or the medicament described herein are designed for delivery to subjects in need thereof by any suitable route. In some embodiments, the composition or the medicament may be in a form of solid, powder, drops, solution, etc.

Another embodiment of the present invention relates to method of prophylaxis and/or therapy of neurological conditions in a subject in need thereof comprising the step of administrating the recombinant AAV vector provided herein to the subject, and the recombinant AAV vector targets GABAergic neurons.

In an embodiment herein, the method of prophylaxis and/or therapy of neurological conditions in a subject further comprises the step of activation of the GABAergic neurons, wherein the activation is selected from the group consisting of chemogenetic activation of the GABAergic neurons by administering a chemical compound, optogenetic activation of the GABAergic neurons by using high frequency laser stimulation (HFLS), and a combination thereof.

In an embodiment herein, the brain condition is selected from the group consisting of epilepsy, Alzheimer's disease (AD), schizophrenia, autism, depression, stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, traumatic brain injury, and a combination thereof. In an embodiment herein, the brain condition is selected from the group consisting of epilepsy, stroke, cancer, and a combination thereof. In an embodiment herein, the brain condition is selected from the group consisting of epilepsy, schizophrenia, autism, depression, and a combination thereof. In an embodiment herein, the brain condition is epilepsy. The vectors and compositions provided herein act broadly on the brain to balance the excitation and inhibition (E/I) in cortex, which offers beneficial effects of treating the above-listed diseases related to E/I imbalance.

In an embodiment herein, the epilepsy is selected from the group consisting of generalized epilepsy, focal epilepsy, pharmacoresistant epilepsy, genetic epilepsy, or a combination thereof. In an embodiment herein, the epilepsy is selected from the group consisting of temporal lobe epilepsy, pharmacoresistant epilepsy, genetic epilepsy, or a combination thereof. In an embodiment herein, the temporal lobe epilepsy is selected from the group consisting of focal epilepsy, pharmacoresistant epilepsy and a combination thereof. Pharmacoresistant epilepsy (also known as medically intractable or refractory epilepsy) is often a chronic, lifelong problem and is associated with significant disease-related costs (both treatment and societal). The present invention provides another option for treating pharmacoresistant epilepsy so as to avoid the high risk of damaging brain functions caused by surgery.

In an embodiment herein, the recombinant AAV vector or the composition according to the invention is administered by an administration method selected from the group consisting of an intramuscular injection, an intravenous injection, an intraperitoneal injection, a subcutaneous injection, a spinal injection, an intraocular injection, and a combination thereof. Via these administration routes, the recombinant AAV vector or the composition according to the invention can penetrate blood-brain barrier (BBB) successfully to infects GABAergic neurons, and thus avoid the use of directly injection into brain tissues that may be too risky and cause undesired brain damages.

In an embodiment herein, the recombinant AAV vector or the composition/medicament according to the invention is administered on a periodic basis; such as once a week; once every 2 weeks; once every 3 weeks; or once a month; or once every 2 months; once every 3 months; or once a year. Due to the long-lasting potentiation of inhibition provided by the invention, the recombinant AAV vector and the composition of the invention can be administrated at long intervals, which is cost effective and may offer conveniences for patients who receive the vector or the composition.

In an embodiment herein, the activation of GABAergic neurons induces long-term enhancement of inhibition onto their output neurons. In an embodiment herein, the long-term enhancement of inhibition lasts from at least about 3 days; about 1 week, about 2 weeks, about 3 weeks, about 1 month, about 2 months, about 3 months, or about 1 year, to at most about 2 years, or even about 5 years after the High Frequency (HF) activation of GABAergic neurons.

In an embodiment herein, the chemical compound for chemogenetic activation comprises clozapine-N-oxide (CNO; or an analogue). Without being bound to theory, it is believed that if CNO or analogues thereof can bind to hM3Dq and activate those receptors, then the neurons will be activated. CNO or analogues thereof can trigger HF firing of the neurons which is similar to the HF activation via activating hM3Dq, and CNO can induce a range of HF firing frequencies (about 10 Hz to about 80 Hz).

In an embodiment herein, the chemical compound is administrated at a dosage of about 0.1 mg/kg to about 7 mg/kg, about 0.5 mg/kg to about 7 mg/kg, about 0.5 mg/kg to about 5 mg/kg, about 0.6 mg/kg to about 4 mg/kg; about 0.6 mg/kg to about 3 mg/kg, about 0.6 mg/kg to about 2 mg/kg based on the weight of the subject to be treated. In some examples, the dosage may be about 0.7 mg/kg, about 0.8 mg/kg, about 1 mg/kg, about 2 mg/kg based on the weight of the subject to be treated. The dosage of the chemical compound is preferably in a range from about 0.5 mg/kg to about 3 mg/kg, and more preferably from about 0.6 mg/kg to about 2.5 mg/kg based on the weight of the subject to be treated. Such dosages are believed to be adequate for activating GABAergic neurons and meanwhile avoid potential side effects, such as sleepy, motionless, and memory loss.

In an embodiment herein, the chemical compound for chemogenetic activation is administrated by an administration method selected from the group consisting of an intramuscular injection, an intravenous injection, an intraperitoneal injection, a subcutaneous injection, a spinal injection, an intraocular injection, orally taken, and a combination thereof.

In an embodiment herein, the high frequency laser (HFL) for optogenetic activation has a frequency in a range of from about 5 Hz to about 200 Hz. In some examples, the HFL for activation has a frequency in a rage of from about 10 Hz to about 150 Hz, from about 10 Hz to about 160 Hz, from about 20 Hz to about 180 Hz, from about 20 Hz to about 160 Hz, from about 30 Hz to about 150 Hz, or from about 40 Hz to about 150 Hz. In some examples, the HFL for activation has a frequency of about 45 Hz, about 50 Hz, about 55 Hz, about 60 Hz, about 65 Hz, about 70 Hz, about 75 Hz, about 80 Hz, about 85 Hz, about 90 Hz, about 95 Hz, about 100 Hz, about 110 Hz, about 120 Hz, about 130 Hz, about 140 Hz or about 150 Hz. By applying HFL having a frequency in a range from about 5 Hz to 200 Hz, it is believed that different types of interneurons will be covered and activated.

In an embodiment herein, from about 1 HFL pulse to about 1000 HFL pulses are applied during the optogenetic activation, for example, 1 HFL pulse, 5 HFL pulses, 10 HFL pulses, 100 HFL pulses, 200 HFL pulses, 400 HFL pulses, 500 HFL pulses, 600 HFL pulses, or 800 HFL pulses. By selecting these number of pulses, it is believed that those neurons that response to the HFLS are selectively activated, while those nonresponsive neurons are left alone. In this way, the side effects are minimized while desired therapeutic effects can be attained.

In an embodiment herein, the HFL is applied for a duration of from about 1 ms to about 10 ms. For example, the duration may be about 2 ms, about 4 ms, about 5 ms, about 6 ms, about 8 ms, or about 9 ms.

In an embodiment herein, the percentage of the GABAergic neurons that are activated by the vector or the method according to the invention ranges from about 5% to about 80%. For example, the percentage of the activated GABAergic neurons in the total GABAergic neurons may be at least about 10%, at least about 20%, at least about 50%, at least about 60, or at least about 70%. It is believed that in certain embodiments herein, the present invention can activate a small portion (e.g., about 20%) of GABAergic neurons, and thereby significantly enhance the potentiation of inhibition.

In an embodiment herein, the method has no influence on astrocytic reactivity in the cortex and locomotion.

Without intending to be limited by theory it is believed that GABAergic neurons maintain the cortex in balanced excitation and inhibition, and dysfunction of GABAergic neurons can result in several disorders, including but not limited to epilepsy. In temporal lobe epilepsy (TLE), the excitation can easily propagate from one site to another, indicating too much connectivity among the excitatory synapses. It is believed that restoring the balance between excitation and inhibition is one of the strategies for treating epilepsy. The present invention demonstrates that partial transduction of the inhibitory neurons in the whole brain leads to sufficient therapeutic impacts in inhibiting the epileptic attacks.

The present invention surprisingly finds that high frequency laser stimulation (HFLS) of GABAergic neurons optogenetically induces an enhanced inhibition in the neocortex, showing suppression of spontaneous firings and response to the imminent natural stimulus. In addition, the present invention shows that chemogenetically induced high-frequency firing of GABAergic neurons leads to the enhancement of cortical inhibition, and such enhanced inhibition could be useful for treating neurological disorders that distribute widely in the brain or involve the entire nervous system.

Surprisingly, it is believed that the present invention can specifically activate GABAergic neurons in the auditory cortex (AC) with optogenetic and chemogenetic technology. Accordingly, an embodiment of the invention herein relates to a method of prophylaxis and/or therapy of neurological conditions in a subject in need thereof including the step of administrating a recombinant AAV vector or a composition comprising the same as described herein to a subject. Typically, the recombinant AAV vector then targets GABAergic neurons.

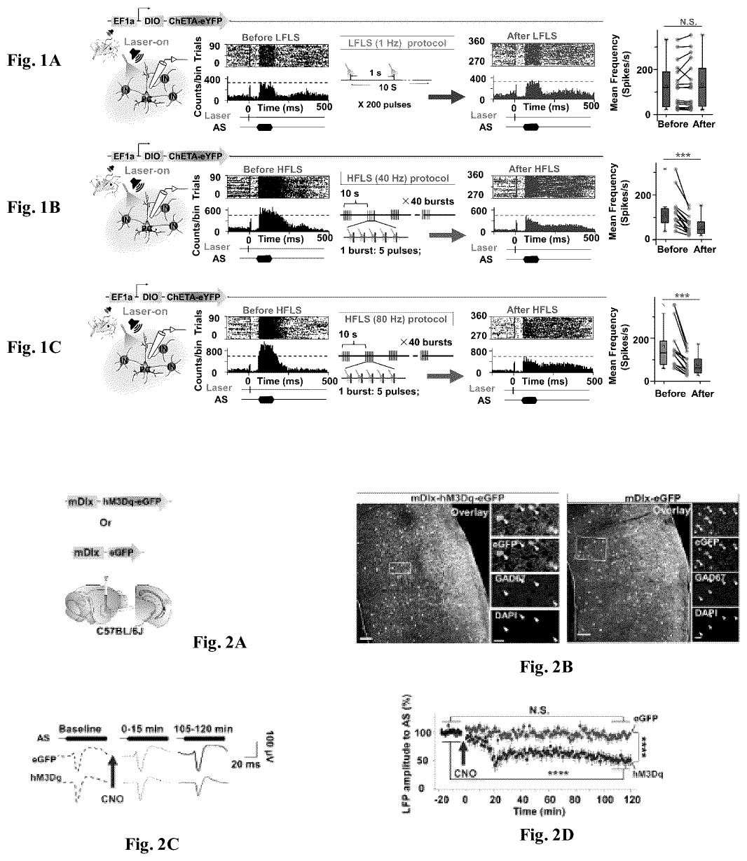

Without intending to be limited by theory, it is believed that activated GABAergic neurons may impose inhibitions on other cortical neurons, thus suppressed neuronal responses to the forthcoming inputs such as the AS in the present invention. The HFLS of GABAergic neurons potentiates the strength of inhibition, and the potentiated inhibition is long-lasting. Our in vivo recording results in FIG. 1 show that HFLS of GABAergic neurons potentiates the inhibition while the same number of pulses in low-frequency laser stimulation (LFLS) does not. Without intending to be limited by theory, it is believed that the key factor here is high frequency. It is believed that the mechanism of how HFLS of GABAergic neurons induces enhancement of inhibition may relate to the co-release of neuropeptide(s) and GABA.

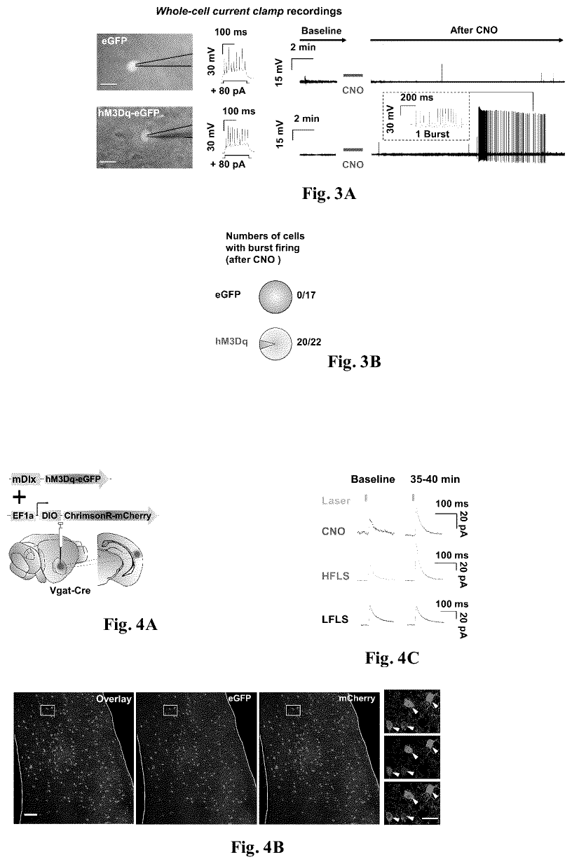

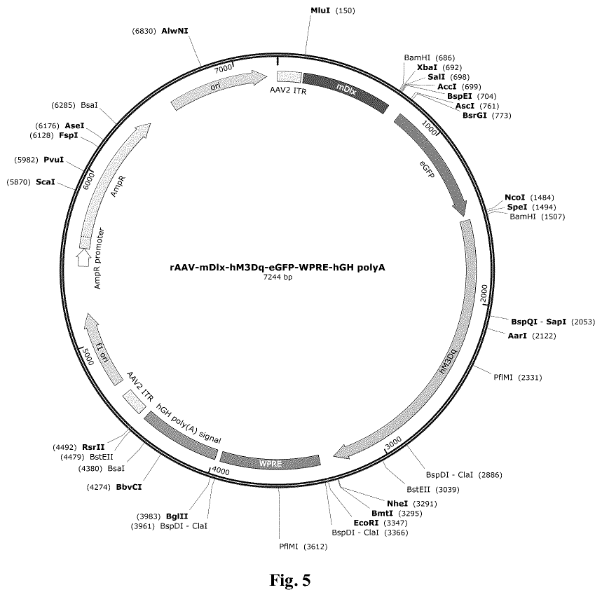

Chemogenetic excitation induces high-frequency firing of GABAergic neurons and generates potentiated and prolonged suppression of neuronal responses (see, for example, FIG. 2 and FIG. 3). Using our self-designed viral vector that penetrated BBB, the present invention successfully infects GABAergic neurons in various brain regions after intravenous administration

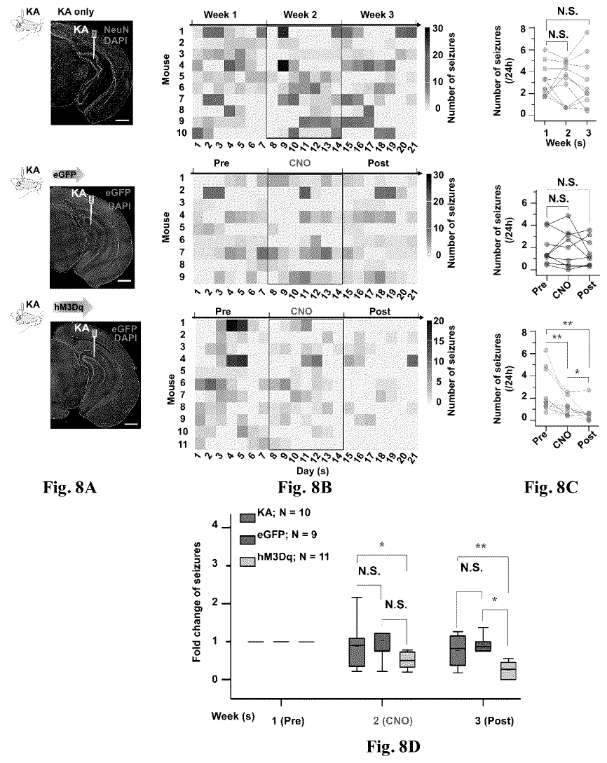

In the present invention, the intrahippocampal KA-injection-induced epilepsy model, which is regarded as the most reliable temporal lobe epilepsy model is adopted (see the Materials and Methods section in the example). By activation of hM3Dq expressed on the inhibitory neurons, the numbers of seizures significantly reduce as shown in the data of FIG. 8. Partial transduction of the inhibitory neurons in the whole brain leads to sufficient therapeutic impacts in inhibiting the epileptic attacks.

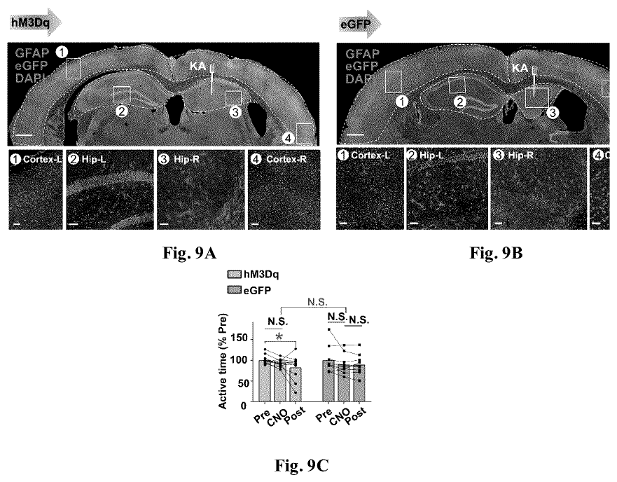

Both the clinical trials and preclinical studies of AAV-based gene therapy for neural disorders in prior art face several obstacles, including the therapeutic potency, AAV vector immunogenicity, and potential genotoxicity. Surprisingly, in the present invention, no astrocytic reactivity is found in the cortex where the virus infected many neurons after the chemogenetic therapy indicated by the astrocyte differentiation marker: GFAP (FIG. 9). No significant behavioral effects, including motionlessness and locomotion, are detected after CNO administration (FIG. 9C).

In the present invention, the inventors successfully restrict the hM3Dq in the inhibitory neurons on a large scale. The delivery of CNO has a robust anti-seizure effect in chronic epilepsy. With great caution in manipulating inhibitory neurons in future preclinical or clinical trials, the updated therapeutic AAV should be safer and more considerate.

Another embodiment of the invention relates to use of a recombinant AAV vector in the manufacture of a medicament of prophylaxis and/or therapy of neurological conditions in a subject in need thereof, in which the recombinant AAV vector includes PHP.eB, a sequence for introducing the expression of human M3 muscarinic DREADD receptor coupled to Gq (hM3Dq); and an inhibitory neuron-specific Distal-less homeobox (Dlx) gene enhancer sequence. The features of the recombinant AAV vector described herein are applicable for this embodiment.

In an embodiment herein, the medicament is in a formation suitable for being delivered directly through blood vessels. In an embodiment herein, the medicament is a form selected from the group consisting of solid, powder, drops, and solution.

Another embodiment of the invention relates to a method of delivering a therapeutic agent to specific neurons in a subject comprising the step of administrating the recombinant AAV vector provided herein to the specific neurons.

In an embodiment, the therapeutic agent for delivering is one or more selected from the group consisting of small peptide, agonist, nanoparticle, antibody, nucleic acid, mRNA, and a combination thereof. For example, the small peptide is selected from the group consisting of CCK-4; CCK-8s, SOM, VIP, or their analogues; the agonist is selected from the group consisting of CCK-4; CCK-8s, SOM, VIP, or their analogues; the nanoparticle is selected from the group consisting of CCK-4; CCK-8s, SOM, VIP, or their analogues; the antibody is selected from the group consisting of CCK-4; CCK-8s, SOM, VIP, or their analogues; the nucleic acid is selected from the gene or sequence consisting of CCK-4; CCK-8s, SOM, VIP, or their analogues; the mRNA is selected from the sequence consisting of CCK-4; CCK-8s, SOM, VIP, or their analogues.

### EXAMPLES

### Materials and Methods

### Mice

The following mouse lines are used in this study: Vgat-ires-Cre (^{Slc32a1tm2(Cre)Lowl}/J with a C57BL/6J background, Vgat-Cre, Jackson Laboratory) and C57BL/6J mice. All experimental procedures are approved by the City University of Hong Kong Animal Subjects Ethics Sub-Committees.

### Surgery and Virus Injections

Mice (The Jackson Laboratory; Bar Harbor, Maine; US; https://www.jax.org/; Stock No. 016962) are anesthetized with pentobarbital (100 mg/kg) supplemented with atropine (0.05 mg/kg, Sigma). The anesthetized mice are head-fixed using a stereotaxic device (RWD Life Science, Shen Zhen, China, https://www.rwdstco.com/). The dura mater is opened, and a craniotomy is performed to access the auditory cortex (AC, −2.0 to −3.0 mm posterior to the bregma and −4.0 to −4.3 mm to the midline). A double loxP-flanked (DIO) Cre-dependent adeno-associated viral (AAV) vector expressing channelrhodopsin-2 (ChETA) fused with an enhanced yellow fluorescent protein (eYFP) (AAV-EF1α-DIO-ChETA-eYFP, University of North Carolina, Vector Core) is injected into the ACs of the Vgat-Cre mice. Four AC locations are each injected with 300 nL of the AAV vector (8E+12 VG/mL, VG: vector genome) at a speed of 30 nL/min (Nanoliter Injector, World Precision Instruments; UK; https://www.wpiinc.com/). The same procedures are adopted for the virus injection in the C57BL/6J mice.

This invention uses the blood-brain barrier (BBB)-penetrating AAV-PHP.eB. The AAV comprising a woodchuck hepatitis virus post-transcriptional regulatory element (WPRE) are designed to express an enhanced green fluorescent protein (eGFP) and human M3 muscarinic DREADD receptor coupled to Gq (hM3Dq) under the control of the inhibitory neuron-specific Distal-less homeobox (Dlx) gene enhancer sequence (rAAV-mDlx-hM3Dq-eGFP-PHP.eB-WPREs or rAAV-mDlx-eGFP-PHP.eB-WPREs).

### Optogenetics and In Vivo Local Field Potential (LFP) and Unit Recordings

Mice (Jackson Laboratory; Bar Harbor, Maine; US; https://www.jax.org/; Stock No. 016962) are prepared for in vivo recordings. Animals are anesthetized with urethane sodium (Sigma; 2 g/kg) by intraperitoneal injection (i.p.) and head-fixed using a stereotaxic device (Narishige, Japan; https://usa.narishige-group.com/). Atropine sulfate (0.05 mg/kg) is administered 15 min before anesthesia to inhibit tracheal secretions. A local anesthetic (xylocaine, 2%) is liberally applied to the incision site during surgery. The dura mater is opened, and a craniotomy is performed to access the AC (−2.0 to −3.0 mm posterior to the bregma and −4.0 to −4.3 mm to the midline).

Then, recording electrode arrays (0.5-1.0 MΩ, FHC Inc.) or glass electrode (5-7 MΩ, WPI) pulled from the P-97 Pipette Puller (Sutter Instrument; CA, US, https://www.sutter.com/) are inserted into the AC. A laser fiber is placed on the surface of the AC, where the virus is infused. Electrodes are positioned with a stepping-motor micro driver that is controlled from outside the soundproofed chamber. Recording electrodes are attached to a 16-channel, head-stage pre-amplifier. Signals are pre-amplified, filtered with a bandwidth of 1-5,000 Hz, and stored in the TDT workstation (OpenEX, TDT, Florida, United States, https://www.tdt.com/). The auditory stimulus (AS), a noise burst, is digitally generated by the TDT workstation and delivered through a coupled electrostatic speaker (50-105 dB, Sound Pressure Level, SPL). A pulse at a 473-nm wavelength is provided by the laser generator (NEWDOON, Changchun, China; http://www.newdoon.com) and controlled by the TDT workstation. The intensity of the laser is tested and monitored around 10 mW.

The input-output curve between the sound intensity (60-95 dB, SPL) and the amplitude of the LFP is measured. The intensity that evoked 50% of the maximum response is adopted as the sound intensity. The input-output curve between the laser power (0-15 mW) and the amplitude of the LFP are measured to help select the laser power applied in the examples. The laser power that evoked 50% to 70% of the maximum response is adopted as the stimulation power in every experiment.

Viral expression is confirmed by the laser-induced suppressive effect on the neuronal response to the AS. The interval between the laser and AS is changed to examine the suppressive effect of the laser activation of inhibitory neurons on the neuronal response to the forthcoming AS and to identify the best interval for the suppression effect. The LFP traces are calculated with Matlab (MathWorks.Inc, Natick, US; https://ww2.mathworks.cn/) and plotted over time.

Spontaneous neuronal activities, neuronal responses to the laser pulse, and neuronal responses to the laser and AS combination are recorded as units and LFP (0.1 Hz) for 15 min (see Examples 1 and 2). Single-pulse lasers (5-10 mW, 5-ms duration) are delivered in 0.1-Hz repetitions 50 ms before the AS, and recordings are acquired for 15 min (baseline) and 60 min after the intervention of either LFLS (200 pulses at 1 Hz) or HFLS (5 pulses at 40 Hz, repeated in 40 trials at 0.1 Hz; or 5 pulses at 80 Hz, repeated in 40 trials at 0.1 Hz) (See, FIG. 1, Example 2). The response to 5 consecutive pulses of the laser at different frequencies (1-120 Hz, repeated in 20 trials at 0.1 Hz) is examined (see Example 2).

### AAV-PHP.eB Production

The viral tools are all packaged by BrainVTA (BrainVTA Co., Ltd., Wuhan, China). Briefly, the AAV-mDlx capsids are built by replacing the mDlx gene with EF1α sequence in the rAAV-EF1α-hM3Dq-eGFP-WPREs or rAAV-EF1α-eGFP-WPREs. The main pAAV plasmid contains AAV2 Inverted Terminal Repeats (ITRs). The AAV2 comprises a nucleic acid sequence according to SEQ ID NO. 1 or a nucleic acid with an identity of from at least about 80%, at least about 90%, about 95%, about 96%, about 97%, about 98% or about 99% to about 100% of SEQ ID NO. 1. Separate Rep/Cap plasmid and the helper Plasmid provides components of the viral replication machinery and the capsid proteins of selected AAV serotypes. After HEK293 cell lysis, viral particles are purified by CsCl gradient ultracentrifugation. And rAAVs are tittered by quantitative polymerase chain reaction (qPCR).

### Clozapine-N-Oxide (CNO) Administration and In Vivo Recordings

In vivo recordings are performed in the ACs of mice using the same surgery setup described above. The spontaneous firing is recorded for 15 min (baseline) followed by the neuronal responses to AS. Then, the inventors intraperitoneally administrate CNO (2 mg/kg) to the mice that are previously injected with either rAAV-mDlx-hM3Dq-eGFP-PHP.eB-WPREs (2.05E+11 VG/mL) or rAAV-mDlx-GFP-PHP.eB-WPREs (2.07E+11 VG/mL), and the recordings are continued for an additional 2 h.

### In Vivo LFP Analysis

For each time point, an average of six evoked responses is used. The inventors average the LFP amplitudes every six trials (MATLAB), which represents a 1-min recording. The representative waveforms are also plotted with MATLAB (MathWorks.Inc, Natick, US; https://ww2.mathworks.cn/). Baseline recordings are taken 15 min and 60 min after 10-s intervals of HFLS or LFLS or 120 min after CNO (i.p.) administration. The results are exported into Excel for offline analysis and plotting.

Group data are presented as the mean f standard error of the mean (SEM). All experiments are reproduced using biological replicates. Attempts at reproduction are successful. Three to 10 mice are used per condition for each experiment. The AS and optogenetics timing activation are randomized. Male or female mice are randomly selected for each experiment. For all electrophysiology experiments, “n” refers to the number of recording sites per condition, and “N” referred to the number of animals. The analysis is conducted in a blinded manner. For experiments involving two independent variables and where n is too small to determine distribution, a normal distribution is assumed and two-way analysis of variance (ANOVA) with Tukey's post hoc test is performed with SPSS or Excel. Group data shown in all figures are drawn with Origin Pro 8.5.

### Current-Clamp Cortical Slice Recordings and CNO Application

Briefly, anesthetized mice receive transcardial perfusion withN-methyl-D-glucamine (NMDG)-aCSF (92 mM NMDG, 2.5 mM KCl, 1.2 mM NaH₂PO₄, 30 mM NaHCO₃, 20 mM N-2-Hydroxyethylpiperazine-N-2-Ethane Sulfonic Acid (HEPES), 25 mM glucose, 2 mM thiourea, 5 mM Na-ascorbate, 3 mM Na-pyruvate, 0.5 mM CaCl₂·4H₂O, and 10 mM MgSO₄·7H₂O; adjusted pH to 7.3-7.4 with concentrated HCl), and the brain is gently and rapidly extracted from the skull and then cut into 300 μm-thick sections with a vibratome (Leica VT1200S). Slices are transferred into NMDG-aCSF for 10 min at 32-34° C. to allow protective recovery and then transferred to room-temperature HEPES-aCSF (92 mM NaCl, 2.5 mM KCl, 1.2 mM NaH₂PO₄, 30 mM NaHCO₃, 20 mM HEPES, 25 mM glucose, 2 mM thiourea, 5 mM Na-ascorbate, 3 mM Na-pyruvate, 2 mM CaCl₂·4H₂O, and 2 mM MgSO₄·7H₂O; adjusted pH to 7.3-7.4) for at least 1 h before recording.

Brain slices are then bathed in room-temperature recording aCSF (124 mM NaCl, 2.5 mM KCl, 1.2 mM NaH₂PO₄, 24 mM NaHCO₃, 5 mM HEPES, 12.5 mM glucose, 2 mM CaCl₂·4H₂O and 2 mM MgSO₄·7H₂O, ˜25° C.) and whole-cell recordings are made at the auditory cortex using Multiclamp 700B amplifier (Molecular Devices, www.moleculardevices.com, Menlo Park, California, USA) and Digital 1440A digitizer (Molecular Devices, www.moleculardevices.com, Menlo Park, California, USA). Patch pipettes with resistance between 5-7 MΩ are pulled from borosilicate glass (WPI, World Precision Instruments; UK; https://www.wpiinc.com/) with a Sutter-87 puller (Sutter Instrument; CA, US, https://www.sutter.com/). The internal solution contains: 130 mM K-Gluconate, 10 mM NaCl, 10 mM HEPES, 1 mM EGTA, 3 mM Mg-ATP, 2 mM Na-GTP, and 0.133 mM CaCl₂); adjusted pH to 7.3 with 1M KOH; 290-300 mOsm. Similarly, recordings are made after Giga-Ohm seal formation holding at −65 to −70 mV and terminated if Rs changes more than 20%. Recording neurons are selected after the observation of eGFP under the Olympus microscope equipped with a far-infrared laser. Before CNO application, neurons are clamp at −65 to −70 mV for 5 min. 0.3 mL CNO (0.2 mg/mL) is gently infused into the recording chamber (5-7 mL recording aCSF) and recorded for at least 20 minutes. The firing rate is analyzed with Clampfit 11.2 (Molecular Devices, www.moleculardevices.com, Menlo Park, California, USA). Action potentials are identified by the Threshold Search and bursts are selected to count firing rate only the spikes numbers more than 5 within each burst. The highest firing burst is selected from each neuron.

### Intrahippocampal KA Injection Induced Chronic Epileptic Mouse Model

To test the hypothesis that the enhanced inhibition could rebalance the inhibition and excitation of the epileptic brain circuit, an intrahippocampal KA-injection induced epilepsy model in mice based on a well-established model by previous investigators (Bielefeld, et al., A Standardized Protocol for Stereotaxic Intrahippocampal Administration of Kainic Acid Combined with Electroencephalographic Seizure Monitoring in Mice, Frontiers in Neuroscience, Vol. 11, pp. 1-9, 2017; Gröticke, et al., Behavioral alterations in a mouse model of temporal lobe epilepsy induced by intrahippocampal injection of kainate, Exp. Neurol., Vol. 213, pp. 71-83, 2008; Raedt et al., Seizures in the intrahippocampal kainic acid epilepsy model: characterization using long-term video-EEG monitoring in the rat, Acta Neurol Scand, Vol. 119, pp. 293-303, 2009) is adopted. Spontaneous seizures three weeks post surgeries are observed. Seizures are classified into five stages according to Racine's scale: 1) mouth and facial movements; 2) head nodding; 3) forelimb clonus; 4) rearing; and 5) rearing and falling (Luttjohann, et al., Physiol. Behav., Vol. 98, pp. 579-586, 2009). Only stages 3-5 seizures are included in the data processing herein. The number of seizures for the entire 24 hours is calculated with software. Without any intervention, each animal's daily spontaneous seizures are monitored for 3 weeks. No decrease in the frequency of epileptic behaviors is observed within 3 weeks (FIG. 8A, compared to week 1, N=8 mice; paired two-tailed 1-test, N.S.).

### Intra-Venous AAV-PHP.eB and Intrahippocampal Kainic Acid (KA) Injection in Mice

C57BL/6J male mice (6-7 weeks old) are anesthetized with pentobarbital (100 mg/kg) supplemented with atropine (0.05 mg/kg, Sigma). The anesthetized mice are head-fixed using a stereotaxic device (RWD Life Science; Shen Zhen, China; https://www.rwdstco.com/). The skull near the lambda is gently removed to expose the brain vessel for rAAV-mDlx-hM3Dq-eGFP-PHP.eB-WPREs injection. The PHP.eB serotype virus, which readily crosses the BBB for brain transduction, is produced using standard production methods. A glass pipette tip (at an angle of about 300 to about 45°) is used to slowly touch the surface of the transverse venous sinus with the injection pump running at a slow speed (100 nL/min). After the glass pipette tip penetrates the vessel, the injection speed is increased to 1,000 nL/min. A total of 50 μL of the virus (1.5E12 VGs/kg of body weight, VG, vector genome) is injected. The same procedures are adopted for injection of the rAAV-mDlx-eGFP-PHP.eB-WPREs control virus.

After viral injection, 650 nL KA (0.3 mg/mL) is administered at the CA1 (−2.06 mm posterior to the bregma, −1.80 mm to the midline, and −1.60 mm to the dura) at a speed of 30 nL/min. For the KA-only group, the C57BL/6J male mice (6-7 weeks old) received 650 nL KA (0.3 mg/mL). Upon recovering from the anesthesia, the mice are monitored for at least an hour for any signs of acute epileptic seizures before being placed back into their cages for routine monitoring.

### Observation and Assessment of Epilepsy and Spontaneous Locomotion Detection in Mice

The animals are housed separately under a standard 12-h light/dark cycle and stable temperature (23-25° C.) with full access to food and water. The mice injected with KA and rAAV-mDlx-hM3Dq-eGFP-PHP.eB-WPREs or rAAV-mDlx-eGFP-PHP.eB-WPREs are kept under continuous video monitoring (24 h per day) using a 360-smart camera at least for 3 weeks after surgery.

The inventors developed software based on deep learning architecture (Pytorch and Python development environment) to accomplish automatic epileptic ictal recognition. The mice seizure recognition system (MSRS) is composed of three main parts, i.e., Video Extraction, Frame Tagging, and Seizure Prediction. First, the histogram equalization is proposed to enhance the contrast of video frames by equalizing pixels of different grayscale in gray frames. The difference between three frames (previous frame, current frame, and last frame) is computed, and then a threshold k is used to detect the noise in the difference. If the difference in the same pixel of the two frames is less than k, then the difference is treated as noise and decreased to 0 (greyscale value). Otherwise, the difference is sharpened to 255 (greyscale value). A summation of each pixel value for the difference is used to decide whether the animal moves between these three frames. If the animal does not, a video interval between these frames is deleted. As a result, some parts of the video are deleted when the animal is sleeping or inactive. Animal movements are analyzed by the program. The active time for each mouse is determined every 24 h. In the next step, histogram equalization is used to enhance the contrast of video frames. Continuous 64 RGB frames stream in 1 min test video are extracted. The inventors predict which video clips show epileptic ictal occurrences based on the epileptic mice databank model trained by fine-tuning our dataset under 3D ResNext-101. Video clips containing suspected seizures are blindly manually confirmed after the original videos are screened out by a computer program.

Based on animal behavioral expressions, seizure intensity is categorized into five stages according to the Racine scale described above. Only stages 3-5 are considered in the examples. For each mouse, the seizures are recorded for 7 days as the baseline. The number of seizures within 24 h per mouse is quantified with software, as previously described. Then, the mice are administered CNO in their drinking water (5 mg/kg/day, about 5-8 mL/day/mouse) for 7 days, and their behaviors are recorded during this time. The mice are monitored for seizures for another 7 days after the CNO treatment. The mice's seizures are evaluated in a blinded manner.

### Immunohistochemistry and Image Acquisition

For immunohistochemistry, mice that previously underwent viral injection are fully anesthetized with pentobarbital sodium (120 mg/kg), and mice are perfused with 30 mL cold phosphate-buffered saline (PBS) and 30 mL 4% (w/v) paraformaldehyde (PFA). Fixed brain tissues are removed and treated with 30% (w/v) sucrose in 4% PFA at 4° C. for 2-3 days. Brain tissues are then sectioned at a 30-μm thickness using a cryostat (Leica CM3500, Germany) and preserved with antifreeze buffer (20% [v/v] glycerol and 30% [v/v] ethylene glycol diluted in PBS) at −20° C.

For immunostaining and quantification of viral-infected neurons with inhibitory neurons, serial brain sections are selected for imaging and analysis.

Brain sections are washed three times for 10 min and are then incubated with blocking buffer (10% [v/v] goat serum in PBS with 0.3% [v/v] Triton X-100) at room temperature. After 2 h, the sections are incubated with mouse anti-GAD67 (Millipore, Sigma-aldrich; St. Louis, MO, US; https://www.sigmaaldrich.cn/; MAB5406, 1:500, diluted in blocking buffer), or rabbit anti-NeuN (ab177487, 1:2000) primary antibodies, or mouse anti-GFAP (Santa Cruz, sc-33673, 1:500) primary antibodies with shaking for 36 h at 4° C.

After washing four times with PBS (10 min each), the sections are then incubated with donkey anti-rabbit 647 (Jackson ImmunoStar, 711-605-152) or donkey anti-mouse 594 (Jackson ImmunoStar, 715-585-150) secondary antibodies for 2.5 h at 37° C. Sections are then rinsed with PBS three times before DAPI staining (1:5,000 [v/v], diluted in PBS) and mounting.

All sections are mounted onto slides with 70% (v/v) glycerol in PBS. Image acquisition (10×, 20×, 63×, and 100× magnification) is performed using a Nikon A1HD25 confocal microscope (Nikon, Tokyo, Japan, https://www.nikon.com.cn). The confocal microscope is equipped with a time-delay integration camera, and it performs line scanning for fast image acquisition at a high-resolution fluorescence signal.

### Imaging and Analysis

Images are taken at 20× magnification and are montaged together with the same laser settings of the parallel study. The gain and exposure parameters are optimized for each image. For magnified images showing detailed labeling, z-stack images throughout the tissue are captured. For co-localization analysis, neurons expressing the indicated reporter are counted using only the corresponding color channel. Then, the neurons co-expressing the marker of interest are counted by the Nikon imaging analysis software (NIS-Elements Viewer 5.21). Bright-spot detection is performed in the brain-region fields of interest, with the software under an appropriate fluorescence threshold. A neuron is considered positive for a given marker if the corresponding signal is above the background fluorescence. The ratio of neurons co-expressing both markers over the total number of cells expressing only the reporter is then calculated.

A two-tailed, unpaired Student's t-test is performed to estimate the statistical differences between the indicated test and control groups. The equal variance between the two populations is not tested. At least three biological replicates per data point are included for all quantifications used in the present study.

### Quantifications, Statistics, and Reproducibility

Quantifications are obtained using a minimum of three independent biological replicates. Data collection and analysis are not performed in a blinded manner, but different research groups performed the quantifications. Group data are presented as the mean f SEM. SPSS 25.0 (IBM) and Excel are used to perform statistical analyses, including Student's unpaired 1-test, Student's paired 1-test, one-way ANOVA with Tukey's post hoc test, and two-way RM ANOVA with Tukey's post hoc test. In all figures, asterisks denote significant differences (*P<0.05, **P<0.01, ***P<0.001, ****P<0.0001, and ####P<0.0001). N.S. indicates that a difference is not significant. OriginPro 8.5 (OriginLab Corporation), Inkscape 0.92.3 (Inkscape), and GraphPad Prism 8 (GraphPad Software) are used to perform the graphing. The reference brain map with anatomical abbreviations is accessible on the Allen Brain Atlas website.

### Example 1

Neuronal Responses to the Auditory Stimulus (AS) are Suppressed by Proceeding Activation of Cortical GABAergic Neurons

An in vivo assay is established to quantitatively evaluate the inhibition strength by examining how the activation of the inhibitory neurons suppressed neuronal responses to the forthcoming AS. First, the Cre-dependent AAV virus, AAV9-EF1α-DIO-ChETA-eYFP (the test group) or AAV9-EF1α-DIO-eYFP (the sham control group) is injected (see Surgery and Virus Injections above) in the AC of Vgat-Cre mice to specifically infect GABAergic neurons. Double labeling of glutamic acid decarboxylase 67 (GAD67, a marker for inhibitory neurons) and eYFP (virus reporter: an enhanced yellow fluorescent protein) is performed between the test group and the control group and it is found that most of the eYFP⁺ neurons are colocalized with GAD67 (ChETA-eYFP: 74.02±2.43%, n=13 sections, N=5 mice; eYFP: 75.44±1.98%, n=10 sections, N=5 mice, unpaired two-tailed 1-test, N.S.).

Then, an optic fiber is inserted into the AC to activate the infected GABAergic neurons and a high-impedance glass electrode array recording multiunit responses and LFP is also inserted to perform in vivo recording (see Optogenetics and in vivo LFP and Unit Recordings).

The results show that, in the test group, laser activation of the inhibitory neurons causes inhibition of the neuronal activities, including spontaneous firings and evoked responses to the AS. It is also found that both types of neurons (with or without direct action potentials to the lase stimulation) show suppressed activities after the laser stimulation, and thus they are not discriminated in the following data analysis. The sham control group (DIO-eYFP injected Vgat-Cre mice) shows the same spontaneous firing before and after laser illumination (100 ms before laser: 30.60±3.00, after laser: 32.75±3.34 Hz, n=25 units; paired two-tailed 1-test, N.S.).

For the test group, the preceding laser stimulation significantly suppresses the evoked response to the forthcoming AS. The mean auditory response to the AS only (without laser stimulation) is 152.23±29.22 Hz, while that to the AS that is preceded with laser stimulation reduces to 104.07±20.75 Hz (n=17 units; paired two-tailed 1-test, ***P<0.001). In contrast, the response to the AS shows no change in the sham control group (the mean auditory response to the AS only (without laser stimulation) vs. the mean auditory response to the AS that is preceded with laser stimulation: 154.48±20.90 Hz vs. 164.57±22.01 Hz n=16 units; paired two-tailed 1-test, N.S.).

The inhibition is also reflected in LFP responses to the forthcoming AS in the ChETA expressing mice. The normalized amplitude of the LFP is suppressed from 1.00±0.01 to 0.71±0.01 (ChETA expressing group: n=18 recording sites, N=11 mice; paired two-tailed 1-test, ****P<0.0001) by the preceding laser when the interval (At) between the laser and the AS is 50 ms. In the sham control group, the normalized amplitude of the LFP is remained unchanged even the AS is preceded by the single pulse laser (DIO-eYFP group: laser-off vs. laser-on: 1.00±0.01 vs. 0.99±0.01, n=17 recording sites, N=10 mice; paired two-tailed 1-test, P=0.61, N.S., not significant). Comparing these two groups, the inhibition induced by the laser is clear to see (0.99±0.05 vs. 0.71±0.01, one-way ANOVA with Tukey's post hoc test, ****P<0.0001).

The interval between the laser and the AS (At) is changed (between 20 ms and 160 ms) to examine the lasting effect of the inhibition of the preceding laser stimulation on LFP responses to the AS. A robust inhibition is observed to the AS when the Δt is from 20 to 120 ms (N=8 mice, paired two-tailed 1-test, *P<0.05, **P<0.01, ****P<0.0001, N.S.). According to this inhibition, Δt=50 ms is selected in the next examples. In summary, this example shows that activation of the local GABAergic neurons in the AC inhibits the spontaneous firing and suppressed the responses to the forthcoming natural AS in the AC.

### Example 2

Inhibition Enhanced After High-Frequency Laser Stimulation (HFLS) of GABAergic Neurons

In this example, the inventors examines whether HFLS and LFLS with the same number of pulses to GABAergic neurons have different effects on the neuronal responses to the forthcoming AS in the long term.

The Cre-dependent AAV virus (AAV9-EF1α-DIO-ChETA-eYFP and the sham control AAV9-EF1α-DIO-eYFP) are respectively injected into the AC of Vgat-Cre mice. Neurons infected by the AAV9-EF1α-DIO-ChETA-eYPF followed the burst of the HFLS of 80 Hz have no significant decay at its 5th pulse (one-way ANOVA with Tukey's post hoc test, N.S., ****P<0.0001). Then, how the LFLS and HFLS can impact the inhibition of the single pule laser on the auditory neurons is examined (LFLS: 1 Hz for 200 s, 200 pulses in total, FIG. 1A; HFLS: forty 5-pulse bursts of 40 Hz or 80 Hz, inter-burst-interval, 10 s, 200 pulses in total, FIG. 1B and FIG. 1C).

Unit responses in the AC to the AS, which is preceded by a single pulse laser stimulation, do not change after the LFLS of the inhibitory neurons (FIG. 1A from left to right panels: before and after the LFLS, and group data of firing rate, 107.43±26.20 to 126.42±17.00 Hz, n=17 units, N=11 mice; paired two-tailed t-test; P=0.09, N.S.), indicating no strengthened inhibition. However, unit responses are decreased greatly after the HFLS of the inhibitory neurons (FIG. 1B, from left to right panels: before and after the HFLS, and group data of firing rate, the firing rate reduces from 115.34±19.05 to 61.19±10.25 Hz, n=13 units, N=10 mice, paired two-tailed 1-test, ***P<0.001; FIG. 1C shows similar results), indicating a significantly strengthened inhibition compared with the control virus group (before and after the HFLS: 141.90±21.98 Hz vs. 134.09±21.17 Hz, n=15 units, N=10 mice; paired two-tailed 1-test; N.S.). LFP to the AS, which is preceded by a single laser pulse with an interval of 50 ms, is suppressed after the HFLS of the inhibitory neurons but not after LFLS. Also recorded are the LFP responses to the AS with a preceding laser pulse before and after the LFLS (the DIO-ChETA-eYFP group, n=12 recording sites; N=9 mice) and HFLS (the DIO-eYFP group; n=16 recording sites; N=11 mice; the DIO-ChETA-eYFP group; n=13 recording sites; N=10 mice; two-way RM ANOVA, ****P<0.0001). Mean LFP amplitudes before (−15-0 min) and ˜1 h after the LFLS or HFLS (45-60 min) are recorded. The HFLS of DIO-ChETA-eYFP infected neurons induces a distinct reduction in amplitude of LFP while the HFLS of DIO-eYFP infected neurons or LFLS of DIO-ChETA-eYFP infected neurons not: 101.19±6.10% to 51.11±9.49% vs. 99.99±0.01% to 107.51±2.33% vs. 100.00±5.39% to 102.18±3.42%; one-way ANOVA with Tukey's post hoc test, ****P<0.0001, N.S. The results show that HFLS of local GABAergic neurons enhances their suppressive effect on neural responses to the AS in the cortex. The enhanced inhibition by the HFLS of inhibitory neurons last for 60 min during the recording time, the effect is termed as long term.

### Example 3

Chemogenetic Induction of High-Frequency Firing of GABAergic Neurons

The above examples shows that HFLS of GABAergic neurons enhanced their inhibition in the cortex. The inventors next attempts to confirm that the above inhibition enhancement could be generated chemogenetically in the neocortex. A new BBB-penetrating AAV-PHP.eB is designed. The AAV comprising a woodchuck hepatitis virus post-transcriptional regulatory element (WPRE) is designed to express an enhanced green fluorescent protein (eGFP) and human M3 muscarinic DREADD receptor coupled to Gq (hM3Dq) under the control of the inhibitory neuron-specific Distal-less homeobox (Dlx) gene enhancer sequence (rAAV-mDlx-hM3Dq-eGFP-PHP.eB-WPREs or its sham control rAAV-mDlx-eGFP-PHP.eB-WPREs). The new AAV is injected into the ACs of C57 mice. Then, CNO is administrated to activate the hM3Dq expressed on GABAergic neurons in the AC of C57 mice. To validate the mDlx-promoted virus expression in the C57 mice, the inventors stain GAD67 in the two groups and found that most of eGFP⁺ neurons are immune-positive for GAD67 (FIG. 2B, mDlx-hM3Dq-eGFP: 84.34±2.29%, n=17 sections, N=5 mice; mDlx-eGFP: 84.32±3.23%, n=18 sections, N=5 mice, one-way ANOVA with Tukey's post hoc test, N.S.).

As an application of the long-term enhancement of inhibition after the high-frequency firing of GABAergic neurons, the treatment of temporal lobe epilepsy is adopted in the mouse model. Optogenetic activation of the GABAergic neurons, the first mode the inventors tried, requires a significant number of trials before reaching a treatment protocol. These trials include: 1) identifying the optimal brain area for laser stimulation; and 2) developing an effective stimulation paradigm with optimal intensity, frequency, and the number of pulses in each treatment, the interval between treatments, and the treatment period. Due to the lack of understanding of temporal lobe epilepsy and a long period of 4-8 weeks needed to develop the temporal lobe epilepsy mouse, there is a need for a more straightforward treatment mode, chemogenetics, to activate as many neurons as they are transfected. In the present example, the inventors record these neurons with a patch-clamp in brain slices to confirm that the chemogenetically transfected neurons can be induced into high-frequency firing when CNO is administrated to them. These neurons and their neighboring brain regions are also recorded with metal electrodes under the in vivo preparation.

The result of neuronal response is in accord with the observation with current-clamp recording preparation (FIG. 3B). CNO-mediated activation of hM3Dq receptors result in a robust increase in the spiking rate of the GABAergic neurons and burst firing in some stages (FIG. 3B). The mean highest firing rate in bursts reaches 33.24±2.46 Hz (13 neurons from N=8 mice; ****P<0.0001, paired two-tailed 1-test).

### Example 4

Chemogenetic Activation of GABAergic Neurons Induced Long-Term Enhancement of Inhibition

To understand the potentiation of inhibition after activation of inhibitory neurons, the same in vivo recording preparation as previously described in Examples 1-2 is adopted. The long-term enhanced inhibition is recorded in the display of neural firing activities.

Under in vivo preparation, the multiunit extracellular recordings show that the firing rate initially increases within the first 15 min, decreases after the second 15 min, and recovers at about one hour (hM3Dq: n=24 multi-units; N=7 mice, firing rate increases from 24.39 f 1.51 to 30.97±3.27 in 0-15 min, **P<0.01; decreases to 20.91±1.57 in 30-45 min, **P<0.01; and recovers to 23.42±2.27 in 75-90 min; while the control group, eGFP: n=26 multi-units; N=8 mice, shows no change after the CNO administration; paired two-tailed 1-test). In general, CNO application suppresses neuronal responses. However, it is interesting to note the temporally increased neuronal firing during the first 15 min after the CNO application.

Up to this point, multiunit recording is adopted. Using the spike-sorting software, it is found that new units appear after the CNO application (the number of spike-sorted units increases from 2.73±0.27 to 4.30±0.27 after the CNO injection, ****P<0.0001; n=30 recording sites). The activation of the new units after the CNO application lasts for 25 minutes and returns to the silent mode. The existing units (referred to as baseline units) decrease their firing rate after the CNO application and sustain at the decreased level during the 2-hour period of recording. The new units activated by CNO for <30 min, are inferred to be mostly GABAergic neurons, while the baseline units are all types, but mostly excitatory neurons. The CNO-activated GABAergic neurons (new units) apply inhibition onto baseline units, causing them to decrease their activities.

Compared to the activities of the new units, it is found that the baseline units' activities do not recover even after the new presumably GABAergic neurons recover from the CNO activation (30 min after CNO). One of the explanations for the sustained suppression of the baseline units is the long-term enhancement of inhibition of the GABAergic neurons caused by CNO activation. Then, the maximum firing frequency during the bursts of the new units after CNO application is determined to be 32.63±3.30 Hz (ranged from 11.02 to 106.66, n=44 units). Summarizing the above, it is believed that CNO application activates the infected GABAergic neurons and thus inhibits the activities of the baseline units. As the GABAergic neuron fired in high-frequency bursts inducing enhanced inhibition, the suppression of the neuronal activity of baseline units or total neuronal populations is sustained in the long term during the recording period.

Whether the chemogenetic activation of the GABAergic neurons would suppress neuronal responses to repeated auditory stimuli is further examined. The inventors record 20 min for baseline responses before and 120 min after CNO administration for both the experimental (mDlx-hM3Dq-eGFP) and the control group (mDlx-eGFP; FIG. 2C). Auditory responses in LFP are suppressed in the long term during the 120 min recording for the experimental group, but not for the sham control group (see FIG. 2C for examples and FIG. 2D for group data; mDlx-hM3Dq-eGFP group: 100% to 51.55±7.14%, one-way ANOVA with Tukey's post hoc test, ****P<0.0001; mDlx-eGFP group: 100% to 99.47±3.01%, one-way ANOVA with Tukey's post hoc test, N.S.; between groups: 99.47±3.01% vs 51.55±7.14%, two-way RM ANOVA with Tukey's post hoc test, ****P<0.0001). These data shows that the chemogenetic activation of the GABAergic neurons can suppress neuronal responses in the AC.

### Example 5

Gene Delivery and Functionality of AAV-mDIx-PHP.eB Through Intra-Venous Infusion

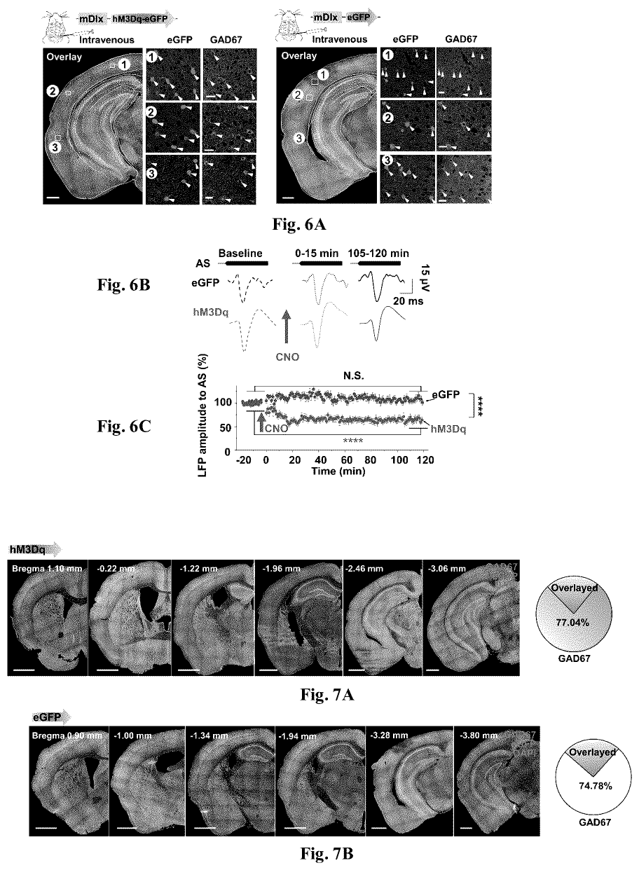

Chemogenetic activation of GABAergic neurons, leading to high-frequency firing, potentiates their inhibition onto their output neurons in the long term. Because of excessive synchronous discharges of excitatory neurons during seizures, the inventors are aiming to chemogenetically activate as many GABAergic neurons as possible to stop the synchronous epileptic activities, and the BBB penetrating AAV-PHP.eB carrying mDlx-hM3Dq-eGFP-PHP.eB or the sham control mDlx-eGFP-PHP.eB are adopted. An anatomical verification of reporter eGFP expression in the entire brains of both mice that received AAV-hM3Dq-eGFP-PHP.eB or its sham control AAV-mDlx-eGFP-PHP.eB is conducted to examine whether the AAV-PHP.eB could penetrate the BBB. Co-localizations of GAD67 and eGFP are analyzed with immunohistochemistry. Most eGFP⁺ neurons in the two groups are GAD67⁺ (FIG. 6A), indicating that the virus is restricted the expression only in the inhibitory neurons under the mDlx promoter control (percentage of GFP and GAD67 co-labeling in the mDlx-hM3Dq-eGFP injected mice: 86.47±0.71%, n=17 sections, N=5 mice; percentage of GFP and GAD67 co-labeling in the mDlx-eGFP injected mice: 85.78±1.18%, n=19 sections, N=5 mice, one-way ANOVA, N.S.). The expression of eGFP is consistently detected through the brain (from Bregma 0.9 mm to −3.8 mm, FIG. 7).

To verify the CNO administration can suppress the response to the AS through the potentiation of inhibition, electrophysiological recordings of neuronal firing and LFP response to the AS before and after the CNO application are performed.

An obvious increase in the spontaneous firing of mDlx-hM3Dq-eGFP-PHP.eB-injected mice (baseline: 42.08±4.25 Hz vs. 0-15 min: 64.02±7.02 Hz) is observed immediately after the CNO intraperitoneal injection (i.p.). The firing rate recovers to the baseline state (105-120 min: 38.64±5.73 Hz, n=23 units, paired two-tailed 1-test, N.S.).

Consistent with the results in Example 4, the response to the AS is suppressed in the experimental group of rAAV-mDlx-hM3Dq-eGFP-PHP.eB but not in the sham group of rAAV-mDlx-eGFP-PHP.eB (FIG. 6B and FIG. 6C, mDlx-hM3Dq-eGFP-PHP.eB: 100.00±0.73% vs. 64.45±1.06%, n=14 recording sites, N=8 mice, two-way RM ANOVA with Tukey's post hoc test, ****P<0.0001; mDlx-eGFP-PHP.eB: 100.00±0.89% vs. 107.68±0.95%, n=11 recording sites, N=7 mice, two-way RM ANOVA with Tukey's post hoc test, N.S.).

### Example 6

Long-Term Suppression of Chronic Epilepsy by Chemogenetic Activation of GABAergic Neurons

KA is injected in the CA1 of the hippocampus to establish the epilepsy model and seizure recognition system (see the Materials and Methods section above, FIG. 8A). Using this model, whether the chemogenetically enhanced inhibition could treat epilepsy is examined. The BBB penetrating AAV-PHP.eB carrying mDlx-hM3Dq-eGFP and its sham mDlx-eGFP are adopted for chemogenetic therapy in this example.

The AAV-mDlx-hM3Dq-eGFP-PHP.eB for the experimental group or AAV-mDlx-eGFP-PHP.eB for the control group are injected into the transverse venous sinus in mice. All mice developed seizures in the 3^{rd} (or 4^{th}) week. Seizures detected by the seizure recognition system (described in the Materials and Methods section above) of each mouse are recorded for 7 days as the baseline (Pre). The number of seizures for the entire 24 hours is calculated with software and manual detection as described above herein. Both groups receive the CNO (5 mg/kg/day, 5-8 m/day/mouse) application (CNO) that is dissolved in the drinking water for 7 days. The behaviors of mice are monitored during the week of the CNO application and a further week after the CNO application with normal drinking water (Post).

The frequency of seizures in the mice with mDlx-hM3Dq-eGFP-PHP.eB decreased after orally CNO delivery (the lower panels of FIGS. 8A, 8B and 8C, Pre: 2.63±0.68; CNO: 1.23±0.25; Post: 0.65±0.27; N=9 mice, paired t-test, *P<0.05, **P<0.01). Meanwhile, the control group mice (mDlx-eGFP-PHP.eB) show spontaneous seizures similarly with before CNO treatment (the middle panels of FIGS. 8A, 8B and 8C, Pre: 1.83±1.38; CNO: 1.95±0.56; Post: 1.54±0.41; N=9 mice, paired 1-test, N.S.). It is striking to note that significant suppression of seizures is observed in the experimental group for the week after the treatment week (Post) in comparison to the week during treatment (CNO), indicating a lasting treatment effect, which is believed due to the long-lasting, enhanced inhibition after the CNO administration.

Finally, anatomical analyses are conducted on all mice from the mDlx-hM3Dq-eGFP-PHP.eB group (N=9 mice) and the mDlx-eGFP-PHP.eB group (N=9 mice), confirming that the intravenously infused AAV/PHP.eB successfully penetrates the BBB and infects the GABAergic neurons in all chronic epilepsy mice.

### Example 7

No Astrocytic Reactivity in Cortex and No Changes in Locomotion after Chemogenetic Therapy

The chronic epilepsy mice model induced by intrahippocampal KA injection is associated with excitotoxicity and astrogliosis in the hippocampus (Chen, et al., J. Neurobiol. Vol. 62, pp. 207-218, 2005). To assess whether the AAV-PHP.eB in this invention introduces huge cytotoxicity and damage to the mice brain, the inventor looked for the immunoactivity of astrogliosis by the GFAP, a marker for the abnormal increase in the number of astrocytes, mainly caused by the cell apoptosis in the brain. In the hippocampus of mDlx-hM3Dq-eGFP-PHP.eB-injected or its sham control mDlx-eGFP-PHP.eB-injected mice, a large amount of eGFP⁺ cells are detected in both hemispheres after 6 weeks of intravenous BBB-penetrating virus and intrahippocampal KA injection in the right hemisphere. Whereas, in the cortex, almost no GFAP⁺ cells are detected where a great deal of eGFP⁺ neurons is located (FIG. 9, for the mDlx-hM3Dq-eGFP-PHP.eB-injected mice, the number of GFAP⁺ cells/mm² in right hippocampus vs. right cortex: 781.11±135.52 vs. 72.08±19.91; n=8 sections, N=4 mice, one-way ANOVA with Tukey's post hoc test, ***P<0.001; the number of GFP⁺ cells/mm²in right hippocampus vs. right cortex: 298.01±40.63 vs. 433.93±30.05; n=8 sections, N=4 mice; one-way ANOVA with Tukey's post hoc test, **P<0.01, ***P<0.001, ****P<0.0001, N.S.).

The behavioral effects after CNO intervention in the two groups are evaluated. Within the mDlx-hM3Dq-eGFP-PHP.eB-injected group or mDlx-eGFP-PHP.eB-injected group, no difference of active time between the two groups is detected during the CNO administration shown in FIG. 9C (one-way ANOVA with Tukey's post hoc test, N.S.).

The side effects are not detected after CNO administration in two groups, from the aspects of astrocytic reactivity and locomotion.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A method of prophylaxis and/or therapy of neurological conditions in a subject in need thereof comprising the step of administrating a recombinant AAV vector to the subject, wherein the recombinant AAV vector targets GABAergic neurons and comprises:
a. PHP.eB;
b. a sequence for introducing the expression of human M3 muscarinic DREADD receptor coupled to Gq (hM3Dq); and
c. an inhibitory neuron-specific Distal-less homeobox (Dlx) gene enhancer sequence.

2. The method of claim 1, wherein the method further comprises the step of activating the GABAergic neurons, wherein the activating is selected from the group consisting of chemogenetic activation of the GABAergic neurons by administering a chemical compound, optogenetic activation of the GABAergic neurons by using high frequency laser stimulation, and a combination thereof.

3. The method of claim 2, wherein the GABAergic neurons are selected from the group consisting of parvalbumin (PV)-GABA neurons, somatostatin (SOM)-GABA neurons, vasoactive intestinal peptide (VIP)-GABA neurons, cholecystokinin (CCK)-GABA neurons, neuropeptide Y (NPY)-GABA neurons, CBPs calbindin (CB)-GABA neurons, calretinin (CR)-GABA neurons, and a combination thereof.

4. The method of claim 1, wherein the condition is selected from the group consisting of epilepsy, Alzheimer's disease (AD), schizophrenia, autism, depression, stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, traumatic brain injury, and a combination thereof.

5. The method of claim 4, wherein the epilepsy is selected from the group consisting of temporal lobe epilepsy, pharmacoresistant epilepsy, genetic epilepsy, or a combination thereof.

6. The method of claim 1, wherein the recombinant AAV vector is administered by an administration method selected from the group consisting of an intramuscular injection, an intravenous injection, an intraperitoneal injection, a subcutaneous injection, a spinal injection, an intraocular injection, and a combination thereof.

7. The method of claim 2, wherein about 5% to about 80% of the GABAergic neurons are activated.

8. The method of claim 2, wherein the chemical compound comprises clozapine-N-oxide or an analogue thereof, or their combinations.

9. The method of claim 2, wherein the chemical compound is administrated by an administration method selected from the group consisting of an intramuscular injection, an intravenous injection, an intraperitoneal injection, a subcutaneous injection, a spinal injection, an intraocular injection, orally taken, and a combination thereof.

10. The method of claim 2, wherein the high frequency laser has a frequency in a range of from about 10 Hz to about 150 Hz.

11. The method of claim 2, wherein the optogenetic activation of the GABAergic neurons induces long-term enhancement of inhibition that lasts from about 1 week to about 2 years.

12. The method of claim 1, wherein the method has no influence on astrocytic reactivity in the cortex and locomotion.

13. Use of a recombinant AAV vector in the manufacture of a medicament of prophylaxis and/or therapy of neurological conditions in a subject in need thereof, wherein the recombinant AAV vector comprises:
a. PHP.eB;
b. a sequence for introducing the expression of human M3 muscarinic DREADD receptor coupled to Gq (hM3Dq); and
c. an inhibitory neuron-specific Distal-less homeobox (Dlx) gene enhancer sequence.

14. The use of claim 13, wherein the recombinant AAV vector specifically targets GABAergic neurons selected from the group consisting of parvalbumin (PV)-GABA neurons, somatostatin (SOM)-GABA neurons, vasoactive intestinal peptide (VIP)-GABA neurons, cholecystokinin (CCK)-GABA neurons, neuropeptide Y (NPY)-GABA neurons, CBPs calbindin (CB)-GABA neurons, calretinin (CR)-GABA neurons, and a combination thereof.

15. The use of claim 13, wherein the recombinant AAV vector targets about 5% to about 80% of the GABAergic neurons.

16. The use of claim 13, wherein the medicament is in a formation suitable for being delivered directly through blood vessels.

17. The use of claim 13, wherein the medicament is administrated by an administration method selected from the group consisting of an intramuscular injection, an intravenous injection, an intraperitoneal injection, a subcutaneous injection, a spinal injection, an intraocular injection, and a combination thereof.

18. The use of claim 13, wherein the medicament is a form selected from the group consisting of solid, powder, drops, and solution.

19. The use of claim 13, wherein the medicament is administrated once a week.

20. The use of claim 13, wherein the neurological conditions are selected from the group consisting of epilepsy, Alzheimer's disease (AD), schizophrenia, autism, depression, stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, traumatic brain injury, and a combination thereof.
